(19) United States
(12) Patent Application Publication
(10) Pub. No.: US 2016/0184435 A1
(43) Pub. Date: Jun. 30, 2016

(54) PHARMACEUTICAL COMPOSITIONS AND DOSAGE FORMS FOR ADMINISTRATION OF HYDROPHOBIC DRUGS

(71) Applicant: Lipocine Inc., Salt Lake City, UT (US)

(72) Inventors: Feng-Jing Chen, Danbury, CT (US); Mahesh V. Patel, Salt Lake City, UT (US); David T. Fikstad, Stanford, CA (US); Huiping Zhang, Malvern, PA (US); Chandrashekar Giliyar, Salt Lake City, UT (US)

(73) Assignee: Lipocine Inc., Salt Lake City, UT (US)

(21) Appl. No.: 14/975,488

(22) Filed: Dec. 18, 2015

Related U.S. Application Data
(63) Continuation of application No. 14/535,536, filed on Nov. 7, 2014, now abandoned.
Application No. 14/535,536 is a continuation of application No. 12/625,284, filed on Nov. 24, 2009, now abandoned.
Application No. 12/625,284 is a continuation of application No. 10/444,935, filed on May 22, 2003.
Application No. 10/444,935 is a continuation-in-part of application No. 09/716,029, filed on Nov. 17, 2000, now Pat. No. 6,982,281.

(51) Int. Cl. A61K 47/22; A61K 47/08; A61K 31/566

(52) U.S. Cl. CPC A61K 47/22; A61K 31/566; A61K 47/08

(57) ABSTRACT

Pharmaceutical compositions and dosage forms for administration of hydrophobic drugs are provided. The pharmaceutical compositions include a therapeutically effective amount of a hydrophobic drug, preferably a steroid; a solubilizer, and a surfactant. The synergistic effect between the hydrophobic drug and the solubilizer results in a pharmaceutical formulation with improved dispersion of both the active agent and the solubilizer. As a result of the improved dispersion, the pharmaceutical composition has improved bioavailability upon administration. Methods of improving the bioavailability of hydrophobic drugs administered to a patient are also provided.

### CROSS-REFERENCE

This application is a continuation of Ser. No. 14/535,536, filed Nov. 7, 2014, which is a continuation of Ser. No. 12/625,284, filed Nov. 24, 2009, which is a continuation of Ser. No. 10/444,935, filed May 22, 2003, which is a continuation-in-part of Ser. No. 09/716,029, filed on Nov. 17, 2000, now issued as U.S. Pat. No. 6,982,281 and a continuation-in-part of Ser. No. 09/877,541, filed Jun. 8, 2001, now issued as U.S. Pat. No. 6,761,903, which is a continuation-in-part of Ser. No. 09/345,615, filed Jun. 30, 1999, now issued as U.S. Pat. No. 6,267,985, and a continuation-in-part of Ser. No. 09/751,968, filed Dec. 29, 2000, now issued as U.S. Pat. No. 6,458,383, which is a continuation-in-part of Ser. No. 09/375,636, filed Aug. 17, 1999, now issued as U.S. Pat. No. 6,309,663, the disclosures of which are incorporated herein by reference in their entireties.

### BACKGROUND OF THE INVENTION

Numerous therapeutic agents are poorly soluble in aqueous medium and present difficult problems in formulating for effective administration to patients. Steroids in particular have very low water solubility and are useful therapeutic agents for a wide variety of medical conditions. Conventional formulations that incorporate these therapeutic agents suffer from several disadvantages such as incomplete or slow dissolution and/or highly variable dissolution profiles. Furthermore, following oral administration, these conventional formulations exhibit low and/or variable absorption. A well-designed formulation must, at minimum, be capable of presenting a therapeutically effective amount of the active substance to the desired absorption site, in an absorbable form.

### SUMMARY OF THE INVENTION

Accordingly, it is a primary object of the invention to address the above-mentioned need in the art by providing a pharmaceutical composition and dosage form for orally administering hydrophobic therapeutic agents.

It is another object of the invention to provide such a composition and dosage form comprising a therapeutically effective amount of a hydrophobic therapeutic agent and a solubilizer.

It is another object of the invention to provide such a composition and dosage form wherein the solubilizer comprises a vitamin E substance, a trialkyl citrate, a lactone, a nitrogen-containing solvent or a combination thereof.

It is still another object of the invention to provide such a composition and dosage form wherein the solubilizer comprises a phospholipid. It is yet another object of the invention to provide such a composition and dosage form wherein the solubilizer comprises a glyceryl acetate, a fatty acid ester of an acetylated glyceride or a combination thereof. It is a further object of the invention to provide such a composition and dosage form wherein the solubilizer comprises a lower alcohol fatty acid ester.

The present invention also encompasses methods of improving the bioavailabilty of active agents, and steroids in particular, in patients through the administration of the claimed pharmaceutical compositions in suitable dosage forms.

Additional objects, advantages and novel features of the invention will be set forth in part in the description which follows, and in part will become apparent to those skilled in the art upon examination of the following, or may be learned by practice of the invention.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

### I. Definitions and Nomenclature

Before the present formulations and dosage forms are disclosed and described, it is to be understood that unless otherwise indicated this invention is not limited to specific dosage forms, solubilizers, or the like, as such may vary. It is also to be understood that the terminology used herein is for the purpose of describing particular embodiments only and is not intended to be limiting.

It must be noted that, as used in the specification and the appended claims, the singular forms “a,” “an” and “the” include plural referents unless the context clearly dictates otherwise. Thus, for example, reference to “a solubilizer” includes a single solubilizer or mixtures of two or more solubilizers, reference to “an additive” refers to a single additive or mixtures of different additives, reference to “an additional active agent” includes a single additional active agent or combinations of two or more additional active agents, and the like.

In this specification and in the claims that follow, reference will be made to a number of terms which shall be defined to have the following meanings:

“Optional” or “optionally” means that the subsequently described circumstance may or may not occur, so that the description includes instances where the circumstance occurs and instances where it does not.

The terms “active agent,” “drug” and “pharmacologically active agent” are used interchangeably herein to refer to a chemical material or compound which, when administered to an organism (human or animal, generally human) induces a desired pharmacologic effect. In the context of the present invention, the terms generally refer to a hydrophobic therapeutic active agent, unless the context clearly indicates otherwise.

By “pharmaceutically acceptable” is meant a carrier comprised of a material that is not biologically or otherwise undesirable.

“Carrier” or “vehicle” as used herein refer to carrier materials suitable for drug administration. Carriers and vehicles useful herein include any such materials known in the art, e.g., any liquid, gel, solvent, liquid diluent, solubilizer, surfactant, or the like, which is nontoxic and which does not interact with other components of the composition in a deleterious manner.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, “treating” a lipid disorder, as the term “treating” is used herein, encompasses both prevention of lipid disorders in a predisposed individual and treatment of lipid disorders in a clinically symptomatic individual.

“Patient” as used herein refers to a mammalian, preferably human, individual who can benefit from the pharmaceutical compositions and dosage forms of the present invention.

The term “vitamin E substance” refers to both vitamin E and derivatives thereof.

By the terms “effective amount” or “therapeutically effective amount” of an agent as provided herein are meant a nontoxic but sufficient amount of the agent to provide the desired therapeutic effect. The exact amount required will vary from subject to subject, depending on the age, weight and general condition of the subject, the severity of the condition being treated, the judgment of the clinician, and the like. Thus, it is not possible to specify an exact “effective amount.” However, an appropriate “effective amount” in any individual case may be determined by one of ordinary skill in the art using only routine experimentation.

### II. The Pharmaceutical Composition

The present invention overcomes the problems associated with the conventional approaches for preparing formulations containing hydrophobic active agents by providing unique pharmaceutical compositions comprising a therapeutically effective amount of an active agent, a solubilizer and, optionally, a dispersing aid, that are more readily dispersed upon mixing with an aqueous medium than those which would be obtained without the particular combination of solubilizer and active agent.

The pharmaceutical compositions described herein contain a hydrophobic therapeutic agent in substantially solubilized form. The compositions improve the bioavailability of the active agent after oral administration and/or improve patient compliance through an easily followed dosing regimen. The compositions described herein preferably contain the active agent, i.e., fenofibrate, in a substantially solubilized form and the effective absorption of the active agent is not dependent on the dissolution of crystalline material of the active agent.

In the art of pharmaceutical formulation, vitamin E substances have been known for their reducing potential and exclusively used as antioxidant in pharmaceutical compositions. The inventors have found, however, that vitamin E substances have unexpected solubilization power toward fenofibrate and other hydrophobic therapeutic agents.

The inventors also have surprisingly found that nitrogen-containing solvents have unexpected solubilization power toward fenofibrate and other hydrophobic therapeutic agents relative to other commonly used non-nitrogen containing solvents such as glycerol, propylene glycol, and polyethylene glycols. With additional research, the inventors have further surprisingly found that replacing one or more of the hydroxyl groups of glycerol and propylene glycol with, for example, a lower alkyl ester, results in a propylene glycol or glycerol fatty acid ester with an unexpectedly high solubilizing power for fenofibrate. Similarly, additional research has yielded other unexpectedly effective solubilizers for fenofibrate including esters of monohydric alcohols such as ethanol and ethylene glycols such as polyethylene glycols with an organic acid such as acetic acid, fatty acids and citric acids. In contrast to most conventional fenofibrate compositions, the present compositions do not require a separate step for the dissolution of crystalline fenofibrate since a significant fraction of fenofibrate is already solubilized in the compositions. In addition, the present compositions are not dependent on lipolysis for the absorption of fenofibrate since the compositions do not require triglycerides or vegetable oils.

### A. Active Agent

The active agent in the present invention is generally hydrophobic in nature (log P greater than 2, P is the intrinsic octanol partition coefficient). Preferred classes of active agents from which the hydrophobic drug may be selected include the following: analgesics, anti-inflammatory agents, anti-helminthics, anti-arrhythmic agents, anti-asthma agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, immunosuppressants, anti-protozoal agents, anti-thyroid agents, anti-tussives, anxiolytics, sedatives, hypnotics, neuroleptics, .beta.-blockers, cardiac inotropic agents, cell adhesion inhibitors, corticosteroids, cytokine receptor activity modulators, diuretics, anti-parkinsonian agents, gastro-intestinal agents, histamine H-receptor antagonists, keratolytics, lipid regulating agents, muscle relaxants, nitrates and other anti-anginal agents, non-steroid anti-asthma agents, nutritional agents, opioid analgesics, sex hormones, stimulants and anti-erectile dysfunction agents.

While this approach may be broadly applicable to many classes of active agents, particularly hydrophobic actives, we have found that drugs in the class of steroids and benzoquinones are particularly effective in this regard.

The following lists set forth exemplary active agents for use in the present invention; those of ordinary skill in the art will readily recognize that suitable active agents may be used in the present invention either alone or in combination.

Steroids are compounds based on the cyclopenta[α]phenanthrene structure. Examples of steroids which have been shown to be suitable for the current invention include those with the androstane structure. Examples of such androstane steroids include cetadiol, clostebol, danazol, dehydroepiandrosterone (DHEA) (also, prasterone or dehydroisoandrosterone), DHEA sulfate, dianabol, dutasteride, exemestane, finasteride, nerobol, oxymethol one, stanolone, stanozolol, testosterone, 17-alpha-methyltestosterone, and methyltestosterone enanthate.

Another group steroids, which have been shown to be suitable, are those based on the cholane or cholesterol structure. Examples of such steroids are brassicasterol, campesterol, chenodeoxycholic acid, clionasterol, desmosterol, lanosterol, poriferasterol, α-sitosterol-, stigmasterol, and ursodeoxycholic acid.

Another suitable class of steroids for use in the present invention are those steroids based on the estrane structure. Examples of such estranes include desogestrel, equilin, 17-alpha-dihydroequilin, 17-beta-dihydroequilin, 17-alpha-estradiol, 17-beta-estradiol (estradiol), ethinyl estradiol, estriol, estrone, levonorgestrel, lynestrenol, mestranol, mibolerone, mifegyne, mifepristone, nandrolone, norethindrone (or norethistrone), norethindrone acetate (or norethisterone acetate), nortestosterone.

Also suitable is the steroid class based on the pregnane structure. Examples of such pregnanes include alfaxalone, beclomethasone, budesonide, clobetasol, clobetasone, corticosterone, desoxycorticosterone, cortisol, cortisone, dihydrocortisone, cyproterone, desonide, dexamethasone, eplerenone, epoxypregnenolone, flumethasone, megestrol, melengestrol, prednisolone, prednisone, pregnanediol, pregnanolone, pregnenolone, allopregnanolone, epiallopregnanolone, progesterone, medroxyprogesterone, spironolactone, and tibolone.

It is to be understood that steroids suitable for the present invention are not limited to those disclosed herein and include any secondary steroids, such as for example, vitamin D.

Steroid esters, such as the acetate, benzoate, cypionate, decanoate, enanthate, hemisuccinate, hexahydrobenzoate, 4-methylvalerate, propionate, stearate, valerate, and undecanoate esters would also be suitable for the present invention.

Examples of suitable benzoquinones include ubiquinones, such as coenzyme Q10, embelin, idebenone [2,3-dimethoxy-5-methyl-6-(10-hydroxydec-yl)-1,4-benzoquinone], pyrroloquinoline quinone, and seratrodast [7-(3,5,6-trimethyl-1,4-benzoquinon-2-yl)-7-phenylheptanoic acid].

Examples of other active agents which may be suitable for this invention include, without limitation: abecarnil, acamprostate, acavir, acebutolol, aceclofenac, acemetacin, acetaminophen, acetaminosalol, acetanilide, acetohexamide, acetophenazine maleate, acetophenazine, acetoxolone, acetoxypregnenolone, acetretin, acrisorcin, acrivastine, acyclovir, adinazolam, adiphenine hydrochloride, adrafinil, adrenolone, agatroban, ahnitrine, akatinol, alatrofloxacin, albendazole, albuterol, aldioxa, alendronate, alfentanil, alibendol, alitretinoin, allopurinol, allylamines, allylestrenol, alminoprofen, almotriptan, alosetron, aloxiprin, alprazolam, alprenolol, amantadine, ambucetamide, amidephrine, amidinomycin, amiloride, aminoarylcarboxylic acid derivatives, aminoglutethimide, aminoglycosides, aminopentamide, aminopromazine, aminorex, amiodarone, amiphenazole, amiprilose, amisuipride, amitriptyline, amlexanox, amlodipine, amodiaquine, amosulalol, amotriphene, amoxapine, amoxicillin, amphecloral, amphetamine, amphomycin, amphotericin, ampicillin, ampiroxicam, amprenavir, amrinone, amsacrine, amyl nitrate, amylobarbitone, anagestone acetate, anastrozole, andinocillin, androstenediol, androstenediol-17-acetate, androstenediol-17-benzoate, androstenediol-3-acetate, androstenediol-3-acetate-17-benzoate, androstenedione, androsterone acetate, androsterone benzoate, androsterone propionate, androsterone, angiotensin, anidulafungin, aniracetam, apazone, apicycline, apoatropine, apomorphine, apraclonidine, aprepitant, aprotinin, arbaprostil, ardeparin, aripiprazole, amikacin, arotinolol, arstiinol, arylacetic acid derivatives, arylalkylamines, arylbutyric acid derivatives, arylcarboxylic acids, arylpiperazines, arylpropionic acid derivatives, aspirin, astemizole, atenolol, atomoxetine, atorvastatin, atovaquone, atropine, auranofin, azapropazone, azathioprine, azelastine, azetazolamide, azithromycin, baclofen, bambuterol, bamethan, barbitone, barnidipine, basalazide, beclamide, beclobrate, befimolol, bemegride, benazepril, bencyclane, bendazac, bendazol, bendroflumethiazide, benethamine penicillin, benexate hydrochloride, benfurodil hemisuccinate, benidipine, benorylate, bentazepam, benzhexol, benziodarone, benznidazole, benzoctamine, benzodiazepine derivatives, benzodiazepine, benzonatate, benzphetamine, benzylmorphine, beperiden, bephenium hydroxynaphthoate, bepridil, betahistine, betamethasone, betaxolol, bevantolol, bevonium methyl sulfate, bexarotene, bezadoxifine, bezafibrate, bialamicol, biapenem, bicalutamide, bietamiverine, bifonazole, binedaline, binifibrate, biricodar, bisacodyl, bisantrene, bisoprolol, bitolterol, bopindolol, boswellic acid, bradykinin, bretylium, bromazepam, bromocriptine, bromperidol, brotizolam, brovincamine, buciclate, bucloxic acid, bucumolol, budralazine, bufeniode, bufetolol, buflomedil, bufuralol, bumetanide, bunitrolol, bupranolol, buprenorphine, buproprion, buspirone, busulfan, butalamine, butarphenol, butaverine, butenafine, butenafine, butidrine hydrochloride, butobarbitone, butoconazole nitrate, butoconazole, butofilol, butorphenol, butropium bromide, cabergoline, calcifediol, calcipotriene, calcitriol, caldiribine, cambendazole, camioxirole, camostat, camposterol, camptothecin, candesartan, candoxatril, capecitabine, caprate, capsaicin, captopril, carazolol, carbacephems, carbamates, carbamezepine, carbapenems, carbarsone, carbatrol, carbenoxolone, carbimazole, carbromal, carbuterol, carisoprodol, carotenes, caroverine, carteolol, carvedilol, cefaclor, cefazolin, cefbuperazone, cefepime, cefoselis, ceftibuten, celcoxib, celecoxib, celiprolol, cephaeline, cephalosporin C, cephalosporins, cephamycins, cerivastatin, certoparin, cetamolol, cetiedil, cetirizine, cetraxate, chloracizine, chlorambucil, chlorbetamide, chlordantoin, chlordiazepoxide, chlormadinone acetate, chlormethiazole, chloroquine, chlorothiazide, chlorpheniramine, chlorphenoxamide, chlorphentermine, chlorproguanil, chlorpromazine, chlorpropamide, chlorprothixene, chlortetracycline, chlorthalidone, cholecalciferol, chromonar, ciclesonide, ciclonicate, cidofivir, ciglitazone, cilansetron, cilostazol, cimetidine, cimetropium bromide, cinepazet maleate, cinnamedrine, cinnarizine, cinolazepam, cinoxacin, ciprofibrate, ciprofloxacin, cisapride, cisplatin, citalopram, citicoline, clarithromycin, clebopride, clemastine, clenbuterol, clidanac, clinofibrate, clioquinol, clobazam, clobenfurol, clobenzorex, clofazimine, clofibrate, clofibric acid, cloforex, clomipramine, clonazepam, clonidine, clonitrate, clopidogrel, clopirac indomethacin, cloranolol, cloricromen, clorprenaline, clortermine, clotiazepam, clotrimazole, cloxacillin, clozapine, cmepazide, codeine methyl bromide, codeine phosphate, codeine sulfate, codeine, colloidal bismuth subcitrate, cromafiban, cromolyn, cropropamide, crotethamide, curcumin, cyclandelate, cyclarbamate, cyclazocine, cyclexedrine, cyclizine, cyclobenzaprine, cyclodrine, cyclonium iodide, cyclopentamine, cyclosporin, cypionate, cyproheptadine, cyproterone acetate, cytarabine, dacarbazine, dalfopristine, dantrolene sodium, dapiprazole, darodipine, decanoate, decitabine, decoquinate, dehydroemetine, delavirdine, delaviridine, demeclocycline, denopamine, deramciclone, descitalopram, desipramine, desloratadine, 3-ketodesogestrel, desomorphine, desoxymethasone, detomidine, dexamphetamine, dexanabinol, dexchlorpheniramine, dexfenfluramine, dexmethylphenidate, dexrazoxane, dextroamphetamine sulfate, dextroamphetamine, dextropropoxyphene, DHEA, diacetate, diamorphine, diazemine, diazepam, diaziquinone, diazoxide, dibromopropamidine, dichlorophen, diclofenac, dicoumarol, didanosine, dideoxyadenosine, diethylpropion, difemerine, difenamizole, diflunisal, digitoxin, digoxin, dihidroergotamine, dihydrocodeine, dihydrocodeinone enol acetate, dihydroergotamine mesylate, dihydroergotamine, dihydrogesterone, dihydromorphine, dihydropyridine derivatives, dihydrostreptomyc in, dihydrotachysterol, dihydroxyaluminum acetylsalicylate, diiodohydroxyquinoline, diisopromine, dilazep, dilevalol, dilitazem, diloxanide furoate, diloxanide, diltiazem, dimefline, dimenhydrinate, dimethisterone, dimetofrine, dimorpholamine, dinitolmide, dioxaphetyl butyrate, dioxethedrine, diphemethoxidine, diphenhydramine, diphenoxylate, diphetarsone, dipivefrin, diponium bromide, dipyridamole, dirithromycin, disopyramide, divalproex sodium, dofetilide, domperidone, donezepil, dopexamine, dopradil, dosmalfate, doxapram, doxazosin, doxefazepam, doxepin, doxycycline, drofenine, dromostanolone propionate, dromostanolone, dronabinol, droperidol, droprenilamine, d-threo-methylphenidate, duloxetine, ebrotidine, eburnamonine, ecabet, ecenofloxacin, econazole nitrate, edavarone, edoxudine, efavirenz, effivarenz, efloxate, eledoisin, eletriptan, elgodipine, ellipticine, emepronium bromide, emetine, enalapril, enanthate, encainide, enlopitat, enoximone, enprostil, entacapone, epanolol, ephedrine, epinastine, epinephrine, epirubicin, epleronone, eposartan, ergocalciferol, ergoloid mesylates, ergotamine, ertapenum, erythromycin, erytlirityl tetranitrate, esaprazole, escitalopram, esmolol, esomeprazole, esonarimod, estazolam, estradiol benzoate, estramustine, estriol succinate, estrone acetate, estrone sulfate, etafedrine, etafenone, ethacrynic acid, ethamivan, ethinamate, ethinylestradiol 3-acetate, ethinylestradiol 3-benzoate, ethinylestradiol, ethionamide, ethisterone (17.alpha-ethinyltestosterone-), ethopropazine, ethotoin, ethoxyphenamine, ethylestrenol, ethylmorphine, ethylnorepinephrine, ethynodiol diacetate, etodolac, etofibrate, etoposide, etoricoxib, etretinate, everolimus, exalamide, examestane, examorelin, ezemitibe, falecalcitriol, famciclovir, famotidine, fantofarone, farapenum, farglitazar, fasudil, felbamate, felodipine, fenalamide, fenbufen, fenbutrazate, fendiline, fenfluramine, fenoldopam, fenoprofen, fenoterol, fenoverine, fenoxazoline, fenoxedil, fenpiprane, fenproporex, fenspiride, fentanyl, fexofenadine, flavoxate, flecainide, flopropione, floredil, floxuridine, fluconazole, flucytosine, fludarabine, fludiazepam, fludrocortisone, flufenamic acid, flunanisone, flunarizine, flunisolide, flunitrazepam, fluocortolone, fluoxetine, flupenthixol decanoate, fluphenazine decanoate, fluphenazine enanthate, fluphenazine, fluproquazone, flurazepam, flurbiprofen, flurogestone acetate, fluticasone propionate, fluvastatin, fluvoxamine, fominoben, formoterol, foscarnet, foscarnet, fosinopril, fosphenytoin, frovatirptan, fudosteine, fumagillin, furazolidone, furazolidone, furfurylmethyl amphetamine, furosemide, gabapentin, gabexate, gaboxadol, galanthamine, gallopamil, gammaparin, ganciclovir, ganglefene, gefarnate, gemcitabine, gemfibrozil, gepirone, gestadene, ghrelin, glatiramer, glaucarubin, glibenclamide, gliclazide, glimepiride, glipizide, gluconic acid, glutamicacid, glyburide, glyceryl trinitrate, glymepiride, granisetron, grepafloxacin, griseofulvin, guaiazulene, guanabenz, guanfacine, halofantrine, haloperidol decanoate, haloperidol, haloxazolam, hepronicate, heptanoate, hexobendine, hexoprenaline, hydramitrazine, hydrazides, hydrochlorothiazide, hydrocodone, hydrocortisone, hydromorphone, hydroxyamphetamine, hydroxymethylprogesterone acetate, hydroxymethylprogesterone, hydroxyprogesterone acetate, hydroxyprogesterone caproate, hydroxyprogesterone, hymecromone, hyoscyamine, ibopamine, ibudilast, ibufenac, ibuprofen, ibutilide, idoxuridine, ifenprodil, igmesine, iloprost, imatinib, imidapril, imidazoles, imipenem, imipramine, imolamine, incadronic acid pergolide, indanazoline, indenolol, indinavir, indomethacin, indoramin, inosinepranobex, inositol niacinate, iodoquinol, ipidracine, iproniazid, irbesartan, irinotecan, irsogladine, isobutyrate, isocaprate esters, isoetharine, isometheptene, isoproterenol, isosorbide dinitrate, isosorbide mononitrate, isosorbide dinitrate, isoxsuprine, isradipine, itasetron, itraconazole, itramintosylate, ivermectin, kallidin, kallikrein, kanamycin, ketamine, ketoconazole, ketoprofen, ketorolac, ketotifen, labetalol, lafutidine, lamifiban, lamivudine, lamotrigine, lanatoside c, lansoprazole, lasofoxifene, leflunomide, leminoprazole, lercanadipine, lesopitron, letrozole, leucovorin, levalbuterol, levallorphan, levetiracetam, levetriacetam, levobunolol, levodopa, levofloxacin, levophacetoperane, levorphanol, lidocaine, lidoflazine, lifibrol, limaprost, linezolid, lintitript, liranaftate, lisinopril, lisuride, lobeline, lobucavir, lodoxamide, lomefloxacin, lomerizine, lomustine, loperamide, lopinavir, loprazolam, loracarbef, loratadine, lorazepam, lorefloxacin, lormetazepam, losartan, lovasatain, lovastatin, loxapine succinate, loxapine, 1-threo-methylphenidate, lumiracoxib, lysine acetylsalicylate, lysozyme, lysuride, mabuterol, mafenide, magnesium acetylsalicylate, malgramostin, mannitol hexanitrate, maprotiline, mazindol, mebendazole, meclizine, meclofenamic acid, mecloxaminepentapiperide, medazepam, medibazine, medigoxin, medrogestone, medroxyprogesterone acetate, mefenamic acid, mefenorex, mefloquin, mefloquine, megestrol acetate, melengestrol acetate, melphalan, mematine, mepenzolate bromide, meperidine, mephenoxalone, mephentermine, mepindolol, mepixanox, meprobamate, meptazinol, mercaptopurine, merropenum, mesalamine, mesalazine, mesoridazine besylate, mesoridazine, metaclazepam, me tamfepramone, metampicillin, metaproterenol, metaraminol, methacycline, methadone hydrochloride, methadone, methamphetamine, methaqualone, methamphetamine, methoin, methotrexate, methoxamine, methsuximide, methylhexaneamine, methylphenidate d-threo-methylphenidate, methylphenidate, methylphenobarbitone, methylprednisolone, methysergide, metiazinic acid, metizoline, metoclopramide, metolazone, metoprolol, metoxalone, metripranolol, metronidazole, mexiletine, mexilitene, metaxalone, mianserin, inibefradil, miconazole, midazolam, midodrine, migitol, milnacipran, milrinone, minoxidil, mirtazapine, misoprostol, mitomycin, mitotane, mitoxantrone, mizolastine, modafinil, mofebutazone, mofetil, molindone hydrochloride, molindone, molsidomine, monatepil, montelukast, monteplase, moprolol, moricizine, morphine hydrochloride, morphine sulfate, morphine, morpholine salicylate, mosapramine, moxifloxacin, moxisylvyte, moxonidine, mycophenolate, nabumetone, nadolol, nadoxolol, nadroparin, nafamostat, nafronyl, naftopidil, nalbuphine, nalidixic acid, nalmefene, nalorphine, naloxone, naltrexone, nandrolone benzoate, nandrolone cyclohexanecarboxylate, nandrolone cyclohexane-propionate, nandrolone decanoate, nandrolone furylpropionate, nandrolone phenpropionate, naphazoline, naproxen, naratriptan, natamycin, nateglinide, nebivalol, nedocromil, nefazodone, nefopam, nelfinavir, nemonapride, neomycin undecylenate, neomycin, neotrofin, nesiritide, n-ethylamphetamine, nevibulol, nevirapine, nexopamil, nicametate, nicardipine, nicergoline, nicofibrate, nicofuranose, nicomorphine, nicorandil, nicotinyl alcohol, nicoumalone, nifedipine, nifenalol, nikethamide, nilutamide, nilvadipine, nimodipine, nimorazole, nipradilol, nisoldipine, nitisonone, nitrazepam, nitrofurantoin, nitrofurazone, nitroglycerin, nizatidine, norastemizole, norepinephrine, norethynodrel, norfenefrine, norfloxacin, norgestimate, norgestrel, norgestrienone, normethadone, normethisterone, normorphine, norpseudoephedrine, nortriptyline, novantrone, nylidrin, nystatin, octamylamine, octodrine, octopamine, ofloxacin, olanzapine, olanzapine, olapatadine, olmesartan, olopatidine, olsalazine, omapatrilat, omeprazole, ondasetron, opium, oprevelkin, orlistat, ornidazole, ornoprostil, oseltamivir, oxaliplatin, oxamniquine, oxandrolone, oxantel embonate, oxaprozin, oxatomide pemirolast, oxatomide, oxazepam, oxcarbazepine, oxfendazole, oxiconazole, oxiracetam, oxolinicacid, oxprenolol, oxycodone, oxyfedrine, oxymetazoline, oxymorphone, oxyphenbutazone, oxyphencyclimine, oxyprenolol, ozagrel, paclitaxel, palonosetron, pantoprazole, papaverine, paracalcitol, paramethadione, parecoxib, pariprazole, paromomycin, paroxetine, parsalmide, pazinaclone, pemoline, penbutolol, penciclovir, penicillin G benzathine, penicillin G procaine, penicillin V, penicillins, pentaerythritol tetranitrate, pentaerythritol tetranitrate, pentapiperide, pentazocine, pentifylline, pentigetide, pentobarbitone, pentorex, pentoxifylline, pentrinitrol, perbuterol, perenzepine, pergolide, perhexiline, perindopril erbumine, perospirone, perphenazine pimozide, perphenazine, phanquinone, phenacemide, phenacetin, phenazopyridine, phencarbamide, phendimetrazine, phenelzine, phenindione, phenmetrazine, phenobarbitone, phenoperidine, phenothiazines, phenoxybenzamine, phensuximide, phentermine, phentolamine, phenyl salicylate, phenylacetate, phenylbutazone, phenylephrinehydrochbride, phenylpropanolamine hydrochloride, phenylpropanolaminehydrochloride, phenylpropyl-methylamine, phenytoin, phloroglucinol, pholedrine, physostigmine salicylate, physostigmine, phytonadiol, phytosterols, piapenum, picilorex, piclamilast, picrotoxin, picumast, pifamine, pilsicaimide, pimagedine, pimeclone, pimecrolimus, pimefylline, pimozide, pinaverium bromide, pindolol, pioglitazone, piperacillin, piperazine estrone sulfate, piperazine derivatives, piperi late, piracetam, pirbuterol, pirenzepine, piribedil, pirifibrate, piroxicam, pitavastatin, pizotyline, plaunotol, polaprezinc, polybenzarsol, polyestrol phosphate, practolol, pralnacasan, pramipexole, praniukast, pravastatin, prazepam, praziquantel, prazosin, pregabalin, prenalterol, prenylamine, pridinol, prifinium bromide, primidone, primipramine, probenecid, probucol, procainamide, procarbazine, procaterol, prochlorperazine, proguanil, pronethalol, propafenone, propamidine, propatyl nitrate, propentoffyline, propionate, propiram, propoxyphene, propranolol, propylhexedrine, propylthiouracil, protokylol, protriptyline, proxazole, pseudoephedrine, purines, pyrantel embonate, pyrazoles, pyrazolones, pyridofylline, pyrimethamine, pyrimidines, pyrrolidones, quazepam, quetiapine, quetuapine, quinagolide, quinapril, quinestrol, quinfamide, quinidine, quinine sulfate, quinolones, quinupritin, rabalzotan, rabeprazole sodium, rabeprazole, racefemine, ramatroban, ramipril, ranitidine, ranolazine, ransoprazole, rasagiline, rebamipide, refludan, repaglinide, repinotan, repirinast, reproterol, reserpine, retinoids, ribavirin, rifabutine, rifampicin, rifapentine, rilmenidine, riluzole, rimantadine, rimiterol, rioprostil, risperidone, ritanovir, ritapentine, ritipenem, ritodrine, ritonavir, rivastigmine, rizatriptan, rociverine, rofecoxib, rohypnol, rolipram, romoxipride, ronifibrate, ropinirole, ropivacaine, rosaprostol, rosiglitazone, rosuvastatin, rotinolol, rotraxate, roxatidine acetate, roxindole, rubitecan, salacetamide, salicin, salicylamide, salicylic acid derivatives, salmeterol, saquinavir, saquinavir, scopolamine, secnidazole, selegiline, semotiadil, sertindole, sertraline, sibutramine, sildenafil, simfibrate, simvastatin, siramesine, sirolimus, sitaxsentan, sofalcone, somotiadil, sorivudine, sotalol, soterenol, sparfloxacin, spasmolytol, spectinomycin, spiramycin, spizofurone, stavudine, streptomycin, succinylsulfathiazole, sucralfate, sufentanil, sulconazole nitrate, sulfacetamide, sulfadiazine, sulfaloxicacid, sulfarside, sulfinalol, sulindac, suloctidil, sulphabenzamide, sulphacetamide, sulphadiazine, sulphadoxine, sulphafurazole, sulphamerazine, sulphamethoxazole, sulphapyridine, sulphasalazine, sulphinpyrazone, sulpiride, sulthiame, sultopride, sultroponium, sumanirole, sumatriptan, sunepitron, superoxide dismutase, suplatast, suramin sodium, synephrine, tacrine, tacrolimus, tacrolimus, tadalafil, talinolol, talipexole, tamoxifen, tamsulosin, targretin, tazanolast, tazarotene, tazobactum, tecastimezole, teclozan, tedisamil, tegaserod, telenzepine, telmisartan, temazepam, teniposide, teprenone, terazosin, terbenafine, terbinafine, terbutaline sulfate, terbutaline, terconazole, terfenadine, terodiline, terofenamate, tertatolol, testolactone, 4-dihydrotestosterone, tetracyclics, tetracycline, tetrahydrocannabinol, tetrahydrozoline, thalidomide, theofibrate, thiabendazole, thiazinecarboxamides, thiocarbamates, thiocarbamizine, thiocarbarsone, thloridazine, thiothixene, tiagabine, tiamenidine, tianeptine, tiaprofenic acid, tiaramide, ticlopidine, tigloidine, tilisolol, timolol, tinidazole, tinofedrine, tinzaparin, tioconazole, tipranavir, tirapazamine, tirofiban, tiropramide, titanicene, tizanadine, tizanidine, tizinadine, tocainide, tolazamide, tolazoline, tolbutamide, tolcapone, tolciclate, tolfenamic acid, toliprolol, tolteridine, tolterodine, tonaberstat, topiramate, topotecan, torasemide, toremifene citrate, toremifene, tosufloxacin, tramadol, tramazoline, trandolapril, tranilast, tranylcypromine, trapidil, traxanox, trazodone, tretoquinol, triacetin, triamcinolone, triampterine, triamterine, triazolam, triazoles, tricromyl, tricyclics, trifluoperazine hydrochloride, trifluoperazine, triflupromazine, trifluridine, trihexyphenidyl hydrochloride, trihexyphenidyl, trimazosin, trimebutine, trimetazidine, trimethoprim, trimgestone, trimipramine, trimoprostil, trithiozine, troglitazone, trolnitrate phosphate, tromethamine, tropicamide, trovafloxacin, troxipide, tuaminoheptane, tulobuterol, tymazoline, tyramine, undecanoate, undecanoic acid, urinastatin, valacyclovir, valdecoxib, valerate, valganciclovir, valproic acid, valsartan, vancomycin, vardenafil, venlafaxine, venorelbine, verapamil, verapimil, vidarabine, vigabatrin, vincamine, vinpocetine, viomycin, viquidil, visnadine, vitamin a derivatives, vitamin a, vitamin b2, vitamin d, vitamin e, vitamin k, voglibose, voriconazole, xaliproden, xamoterol, xanthinol niacinate, xenytropium bromide, xibenolol, ximelagatran, xylometazoline, yohimbine, zacopride, zafirlukast, zafirlukat, zalcitabine, zaleplon, zanamivir, zatebradine, ziconotide, zidovudine, zileuton, zimeldine, zinc propionate, ziprasidone, zolimidine, zolmitrip tan, zolpidem, zonisamide, zopiclone.

An additional active agent may be administered with the active agent included in the compositions and dosage forms of the present invention. It is preferred, however, that the additional active agent is contained within the composition and dosage form. The weight ratio of the primary active agent to the additional active agent may be varied and will depend upon the effective dose of each ingredient. Each active agent contained in the composition or dosage form will be present in a therapeutically effective amount.

Additional active agents may be solubilized or suspended with or without the presence of an additional solubilizer. For those additional active agents that are ionized or ionizable, the formulations described herein may include a buffer to facilitate or maintain the presence of a preferred ionized form of the additional active agent in the formulation.

### B. Solubilizer

The pharmaceutical compositions of the invention also contain a carrier. At a minimum, the carrier must contain a solubilizer. In some instances, the carrier may only contain one solubilizer without any additional components, i.e., additives. Alternatively, the carrier may contain one or more pharmaceutically acceptable additives in addition to the solubilizer.

In one embodiment, a pharmaceutical composition is provided comprising a therapeutically effective amount of an active agent, a solubilizer and a dispersing aid. The solubilizer is present in an amount such that more of the active agent is dispersed; in aqueous medium than that which would be achieved with the same active agent and dispersing aid without the solubilizer. The active agent and the solubilizer act synergistically to improve the dispersibility of the solubilizer itself and the active agent upon dilution in an aqueous media, thus greatly increasing the amount of active agent which can be dispersed in a readily absorbably form. Preferably, the solubilizer is present such that after a 100× dilution of the composition the active agent and/or the solubilizer is at least 30% dispersed in the aqueous phase, with a dispersion of at least 50% being preferred. It is more preferred that the solubilizer, like the active agent is at least 30% finely dispersed in the aqueous phase, with a fine dispersion of at least 50% being most preferred. Thus, the solubilizers as provided herein improve the dissolution profile of a hydrophobic therapeutic agent and thereby the bioavailability of a hydrophobic therapeutic agent.

One type of solubilizer that may be used is a vitamin E substance. This group of solubilizers includes a substance belonging to the group of α-, β-, γ-, δ-, ζ1-, ζ2- and ε-tocopherols, their dl, d and l forms and their structural analogues, such as tocotrienols;-which includes substances with the tocol structure [2-methyl-2-(4,8,12-trimethyltridecyl)chroman-6-ol] or the tocotrienol structure [2-methyl-2-(4,8,12-trimethyltrideca-3,7,11-trienyl)chroman-6-o-1], in particular the all trans-(E,E) tocotrienols. Particularly preferred vitamin E substances include the mono-, di-, trimethyl-tocol derivatives, commonly known as tocopherols, such as α-tocopherol [5,7,8-trimethyl-], β-tocopherol [5,8-dimethyl-], γ-tocopherol [7,8-dimethyl], ζ2-tocopherol [5,7-dimethyl-], δ-tocopherol [8-methyl-], q-tocopherol [7-methyl]; and the corresponding mono-, di-, and trimethyltoctrienol derivatives, commonly known as tocotrienols, such as α-tocotrienol (or ζ1-tocopherol) [5,7,8-trimethyl-], β-tocotrienol (or ε-tocopherol) [5,8-dimethyl], γ-tocotrienol [7,8-dimethyl], and 8-tocotrienol [8-methyl-]. Included are their mixed racemic dl-forms, the pure d- and l-enantiomers and the corresponding derivatives, e.g., esters, produced with organic acids; and mixtures thereof

One skilled in the art can easily identify vitamin E substances that may serve as effective solubilizers by, for example, mixing a particular vitamin E substance with fenofibrate and determining the extent of solubility. Preferred vitamin E substances for use in the present invention include tocopherols, tocotrienols and tocopherol derivatives with organic acids such as acetic acid, propionic acid, bile acid, lactic acid, pyruvic acid, oxalic acid, malic acid, malonic acid, succinic acid, maleic acid, fumaric acid, tartaric acid, citric acid, benzoic acid, cinnamic acid, mandelic acid, polyethylene glycol succinate and salicylic acid. Particularly preferred vitamin E substances include alpha-tocopherol, alpha-tocopherol acetate, alpha-tocopherol acid succinate, alpha-tocopherol polyethylene glycol succinate and mixtures thereof.

Another group of solubilizers are monohydric alcohol esters of organic acids. The monohydric alcohol can be, for example, ethanol, isopropanol, t-butanol, a fatty alcohol, phenol, cresol, benzyl alcohol or a cycloalkyl alcohol. The organic acid can be, for example, acetic acid, propionic acid, butyric acid, a fatty acid of 6-22 carbon atoms, bile acid, lactic acid, pyruvic acid, oxalic acid, malic acid, malonic acid, succinic acid, maleic acid, fumaric acid, tartaric acid, citric acid, benzoic acid, cinnamic acid, mandelic acid and salicylic acid. Preferred solubilizers in this group include trialkyl citrates, lower alcohol fatty acid esters and lactones. Preferred trialkyl citrates include triethyl citrate, acetyltriethyl citrate, tributyl citrate, acetyltributyl citrate and mixtures thereof with triethyl citrate being particularly preferred. Lower alcohol fatty acid esters, as the name implies, comprise a lower alcohol moiety, i.e., containing 2-4 carbon atoms, and a fatty acid moiety of about 6-22 carbon atoms. Particularly preferred lower alcohol fatty acid esters include ethyl oleate, ethyl linoleate, ethyl caprylate, ethyl caprate, isopropyl myristate, isopropyl palmitate and mixtures thereof. Lactones may also serve as a solubilizer. Examples include c-caprolactone, δ-valerolactone, β-butyrolactone, isomers thereof and mixtures thereof

The solubilizer may be a nitrogen-containing solvent. Preferred nitrogen-containing solvents include dimethylformamide, dimethylacetamide, N-alkylpyrrolidone, N-hydroxyalkylpyrrolidone, N-alkylpiperidone, N-alkylcaprolactam and mixtures thereof wherein alkyl is a C₁₋₁₂ branched or straight chain alkyl. Particularly preferred nitrogen-containing solvents include N-methyl 2-pyrrolidone, N-ethyl 2-pyrrolidone or a mixture thereof. Alternatively, the nitrogen-containing solvent may be in the form of a polymer such as polyvinylpyrrolidone.

Another group of solubilizers includes phospholipids. Preferred phospholipids include phosphatidylcholine, phosphatidylethanolamine, phosphatidylserine, phosphatidylinositol, lecithins, lysolecithins, lysophosphatidylcholine, polyethylene glycolated phospholipids/lysophospholipids, lecithins/lysolecithins and mixtures thereof

Another group of preferred solubilizers are glycerol acetates and acetylated glycerol fatty acid esters. Preferred glycerol acetates include acetin, diacetin, triacetin and mixtures thereof, with triacetin being particularly preferred. Preferred acetylated glycerol fatty acid esters include acetylated monoglycerides, acetylated diglycerides and mixtures thereof. In a most preferred embodiment, the acetylated monoglyceride is a distilled acetylated monoglyceride.

In addition, the solubilizer may be a glycerol fatty acid ester. The fatty acid component is about 6-22 carbon atoms. The glycerol fatty acid ester can be a monoglyceride, diglyceride, triglyceride or mixtures thereof. Preferred glycerol fatty acid esters include monoglycerides, diglycerides, medium chain triglycerides with fatty acids having about 6-12 carbons and mixtures thereof. Particularly preferred glycerol fatty acid esters include medium chain monoglycerides with fatty acids having about 6-12 carbons, medium chain diglycerides with fatty acids having about 6-12 carbons and mixtures thereof

The solubilizer may be a propylene glycol ester. Preferred propylene glycol esters include propylene carbonate, propylene glycol monoacetate, propylene glycol diacetate, propylene glycol fatty acid esters, acetylated propylene glycol fatty acid esters and mixtures thereof. Alternatively, the propylene glycol fatty acid ester may be a propylene glycol fatty acid monoester, propylene glycol fatty acid diester or mixture thereof. The fatty acid has about 6-22 carbon atoms. It is particularly preferred that the propylene glycol ester is propylene glycol monocaprylate. Other preferred propylene glycol esters include propylene glycol dicaprylate, propylene glycol dicaprate, propylene glycol dicaprylate/dicaprate and mixtures thereof

Another group of solubilizers are ethylene glycol esters. Ethylene glycol esters include monoethylene glycol monoacetates, diethylene glycol esters, polyethylene glycol esters and mixtures thereof. Additional examples include ethylene glycol monoacetates, ethylene glycol diacetates, ethylene glycol fatty acid monoesters, ethylene glycol fatty acid diesters, and mixtures thereof. Alternatively, the ethylene glycol ester may be a polyethylene glycol fatty acid monoesters, polyethylene glycol fatty acid diesters or mixtures thereof. Again, the fatty acid component will contain about 6-22 carbon atoms. Particularly preferred ethylene glycol esters are those obtained from the transesterification of polyethylene glycol with a triglyceride or a vegetable oil or mixture thereof and include, for example, those marketed under the Labrafil® and Labrasol® names.

Other solubilizers that may be used in the present invention are disclosed in U.S. Pat. Nos. 6,982,281 and 6,761,903, both to Chen et al. Preferred solubilizers that are not vitamin E substances for use in the present invention include fatty acid esters of glycerol, acetylated mono- and diglycerides, fatty acid esters of propylene glycol, trialkyl citrate, glycerol acetate, and lower alcohol fatty acid esters.

### C. Surfactants

The surfactant in the present invention may be any compound containing polar or charged hydrophilic moieties as well as non-polar hydrophobic (lipophilic) moieties; i.e. a surfactant compound must be amphiphilic. Within the context of the present invention, the hydrophilic surfactant can be any hydrophilic surfactant suitable for use in pharmaceutical compositions. Such surfactants can be anionic, cationic, zwitterionic or non-ionic. Mixtures of hydrophilic surfactants are also within the scope of the invention. Similarly, the hydrophobic surfactant can be any hydrophobic surfactant suitable for use in pharmaceutical compositions. Mixtures of hydrophobic surfactants are also within the scope of the invention. Generally, suitable hydrophilic surfactants will have an HLB value greater than about 10 and suitable hydrophobic surfactants will have an HLB value less than about 10. The choice of specific hydrophobic and hydrophilic surfactants should be made keeping in mind the particular hydrophobic therapeutic agent to be used in the composition, and the range of polarity appropriate for the chosen therapeutic agent. With these general principles in mind, a very broad range of surfactants is suitable for use in the present invention.

Examples of surfactants suitable for use in the present invention are disclosed in U.S. Pat. No. 6,294,192 to Patel et al. and U.S. Pat. No. 6,267,985 to Chen et al. Examples of surfactants that may be used in the present invention include polyethoxylated fatty acids such as PEG-8 laurate, PEG-8 oleate, PEG-8 stearate, PEG-9 oleate, PEG-10 laurate, PEG-10 oleate, PEG-12 laurate, PEG-12 oleate, PEG-15 oleate, PEG-20 laurate and PEG-20 oleate; PEG-fatty acid diesters such as PEG-20 dilaurate, PEG-20 dioleate, PEG-20 distearate, PEG-32 dilaurate and PEG-32 dioleate; PEG-fatty acid mono- and di-ester mixtures; polyethylene glycol glycerol fatty acid esters such as PEG-20 glyceryl laurate, PEG-30 glyceryl laurate, PEG-40 glyceryl laurate, PEG-20 glyceryl oleate, and PEG-30 glyceryl oleate; alcohol-oil transesterification products such as PEG-35 castor oil (Incrocas-35), PEG-40 hydrogenated castor oil (Cremophor® RH40), polyoxyl 35 castor oil (Cremophor EL), PEG-25 trioleate (TAGAT® TO), PEG-60 corn glycerides (Crovol M70), PEG-60 almond oil (Crovol A70), PEG-40 palm kernel oil (Crovol PK70), PEG-50 castor oil (Emalex C-50), PEG-50 hydrogenated castor oil (Emalex HC-50), PEG-8 caprylic/capric glycerides (Labrasol®), and PEG-6 caprylic/capric glycerides (Softigen® 767); transesterification products of oils and alcohols; polyglycerized fatty acids such as polyglyceryl oleate (Plurol® Oleique), polyglyceryl-2 dioleate (Nikkol DGDO), and polyglyceryl-10 trioleate. Preferred hydrophilic surfactants include polyglyceryl-10 laurate (Nikkol Decaglyn 1-L), polyglyceryl-10 oleate (Nikkol Decaglyn 1-0), and polyglyceryl-10 mono, dioleate (Caprol® PEG 860); propylene glycol fatty acid esters such as propylene glycol monolaurate (Lauroglycol FCC), propylene glycol ricinoleate (Propymuls), propylene glycol monooleate (Myverol® P-06), propylene glycol dicaprylate/dicaprate (Captex® 200), and propylene glycol dioctanoate (Captex 800); mixtures of propylene glycol esters and glycerol esters such as a mixture of oleic acid esters of propylene glycol and glycerol (Arlacel 186); mono- and diglycerides such as glyceryl monooleate (Peceol), glyceryl ricinoleate, glyceryl laurate, glyceryl dilaurate (Capmul GDL), glyceryl dioleate (Capmul GDO), glyceryl mono/dioleate (Capmul GMO-K), glyceryl caprylate/caprate (Capmul MCM), caprylic acid mono/diglycerides (Imwitor® 988), and mono- and diacetylated monoglycerides (Myvacet® 9-45); sterol and sterol derivatives such as PEG-24 cholesterol ether (Solulan® C-24); polyethylene glycol sorbitan fatty acid esters such as PEG-20 sorbitan monolaurate (Tween® 20), PEG-20 sorbitan monopalmitate (Tween 40), PEG-20 sorbitan monostearate (Tween 60), and PEG-20 sorbitan monooleate (polysorbate 80 or Tween 80); polyethylene glycol alkyl ethers such as PEG-3 oleyl ether (Volpo 3) and PEG-4 lauryl ether (Brij 30); sugar esters such as sucrose monopalmitate and sucrose monolaurate; polyethylene glycol alkyl phenols; polyoxyethylene-polyoxypropylene block copolymers such as Synperonic® PE series (ICI); Pluronicg series (BASF), Emkalyx, Lutrol (BASF), Supronic, Monolan, Pluracare®, and Plurodac; sorbitan fatty acid esters such as sorbitan monolaurate (Arlacel® 20), sorbitan monopalmitate (Span-40), sorbitan monooleate (Span-80), sorbitan monostearate, and sorbitan tristearate; lower alcohol fatty acid esters such as hydrophobic surfactants include ethyl oleate (Crodamol EO), isopropyl myristate (Crodamol IPM), and isopropyl palmitate (Crodamol IPP); ionic surfactants such as sodium oleate, sodium lauryl sulfate, sodium lauryl sarcosinate, sodium dioctyl sulfosuccinate, sodium cholate, sodium taurocholate, lauroyl carnitine, palmitoyl carnitine, and myristoyl carnitine; unionized ionizable surfactants such as free fatty acid, particularly C₆-C₂₂ fatty acids, and bile acids.

Other surfactants for use in the present invention include, without limitation, PEG-400 succinate, PEG 3350, tocopherol polyethyleneglycol (200-8000 MW) succinate, tocopherol polyethylene glycol 400 succinate, tocopherol polyethyleneglycol 1000 succinate (Vitamin E-TPGS, Eastman Chemical Co.), glycerol monolinoleate (Maisine®), propylene glycol monocaprylate (Capryol® 90); caprylocaproyl macrogol-8 glycerides (Labrosol®), glycerol dibehenate (Compritol® 888), glycerol distearate (Precirol®), lauroyl macrogol-32 glycerides (Gelucire® 44/14), and stearoyl macrogol-32 glycerides (Gelucire 50/13).

### D. Additives

Although not always necessary, the compositions of the present invention may also include one or more additional components, i.e., additives. Classes of additives that may be present in the compositions, include, but are not limited to, solvents, absorbents, acids, adjuvants, anticaking agent, glidants, antitacking agents, antifoamers, anticoagulants, antimicrobials, antioxidants, antiphlogistics, astringents, antiseptics, bases, binders, chelating agents, sequestrants, coagulants, coating agents, colorants, dyes, pigments, compatiblizers, complexing agents, softeners, crystal growth regulators, denaturants, dessicants, drying agents, dehydrating agents, diluents, dispersants, emollients, emulsifiers, encapsulants, enzymes, fillers, extenders, flavor masking agents, flavorants, fragrances, gelling agents, hardeners, stiffening agents, humectants, lubricants, moisturizers, bufferants, pH control agents, plasticizers, soothing agents, demulcents, retarding agents, spreading agents, stabilizers, suspending agents, sweeteners, disintegrants, thickening agents, consistency regulators, surfactants, opacifiers, polymers, preservatives, antigellants, rheology control agents, UV absorbers, tonicifiers and viscomodulators. One or more additives from any particular class, as well as one or more different classes of additives, may be present in the compositions. Specific examples of additives are well known in the art.

### III. Determination of Component Amounts and Preparation of Compositions

As will be recognized by those skilled in the art, the amount or percentage of the active agent present in the composition and dosage forms will vary. Thus, for example, the amount of active agent is based, in part, upon the actual need of the patient and can be determined by the attending clinician. In all cases, however, the amount of the active agent present in the composition and dosage forms is an amount such that the active agent is significantly solubilized in the appropriately selected solubilizer or solubilizers so that the aforementioned advantages of the present invention are achieved.

Preferably, the compositions are formulated so as to contain a sufficient amount, i.e., dose, of fenofibrate within a dosage unit, e.g., a capsule. It is preferred that the amount of fenofibrate will be present in the composition so as to provide each dosage form with a unit dosage of from about 40 to about 250 mg, and preferably about 67 to about 200 mg of fenofibrate. It is particularly preferred that the entire amount of fenofibrate is solubilized in the composition. However, it is sometimes necessary to add additional fenofibrate in non-solubilized form when the fenofibrate solubility capacity of a given composition is exceeded. Therefore, it is also an important feature of the present invention that the fenofibrate present in the composition is significantly solubilized. Typically, at least about 50% of the fenofibrate is solubilized in the composition and preferably at least about 75% of the fenofibrate is solubilized in the composition of the dosage form. The dosage form contains fenofibrate solubilized in the composition in an amount of at least about 40 mg, preferably in an amount of at least about 67 mg, and more preferably in an amount of at least about 100 mg.

The amount of solubilizer that can be included in the dosage forms of the present invention is not particularly limited. When the dosage forms are ultimately administered to a patient, however, the amount of any given solubilizer is limited to a bioacceptable amount. Bioacceptable amounts of solubilizers and other components are readily determined by one of skill in the art by using routine experimentation or searching the literature. In some circumstances, it may be advantageous to include amounts of solubilizers far in excess of bioacceptable amounts, for example, to maximize the concentration of the fenofibrate, with excess solubilizer removed prior to providing the composition to a patient. Excess solubilizer may be removed using conventional techniques such as distillation, spray drying, lyophilization or evaporation. Generally, the amount of solubilizer in the composition will be from about 5 wt. % to about 95 wt. %, preferably between about 20 wt. % to about 70 wt. %.

The amount of additional components in the compositions can be determined by one of ordinary skill in the art, according to the desired property or properties to be imparted to the composition. For example, the amount of a suspending agent may be determined by adding gradual amounts of the agent until the desired homogeneity of undissolved drug particles in the composition is achieved. For a colorant, the amount of the colorant may determined by adding small amounts of the colorant until the desired color of the composition is achieved. For a surfactant, the amount of a surfactant may determined by adding gradual amounts of the surfactant until the desired wetting effect or dispersibility of the composition is achieved. The amount of surfactant, when present, in the composition will generally be up to about 80 wt. %, preferably between about 10 wt. % to about 50 wt. %.

The pharmaceutical compositions of the present invention are prepared by conventional methods well known to those skilled in the art. The composition can be prepared by mixing the active agent, the solubilizer, and optional additive according to methods well known in the art. Excess solvent or solubilizer, added to facilitate solubilization of the active agent and/or mixing of the formulation components, can be removed before administration of the pharmaceutical dosage form. The compositions can be further processed according to conventional processes known to those skilled in the art, such as lyophilization, encapsulation, compression, melting, extrusion, balling, drying, chilling, molding, spraying, spray congealing, coating, comminution, mixing, homogenization, sonication, cryopelletization, spheronization and granulation to produce the desired dosage form.

For dosage forms substantially free of water, i.e., when the composition is provided in a pre-concentrated form for administration or for later dispersion in an aqueous system, the composition is prepared by simple mixing of the components to form a pre-concentrate. The compositions comprising solubilized fenofibrate can be further formulated into desirable dosage forms utilizing skills well known in the art. For example, compositions in liquid or semi-solid form can be filled into soft gelatin capsules using appropriate filling machines. Alternatively, the composition can also be sprayed, s granulated or coated onto a substrate to become a powder, granule or bead that can be further encapsulated or tableted if the compositions solidify at room temperature with or without the addition of appropriate solidifying or binding agents. This approach allows for the creation of a “fused mixture,” a “solid solution” or a “eutectic mixture.”

As previously indicated, the compositions may include additional amounts of fenofibrate over the amount that is solubilized in the composition. In such a case, fenofibrate can be partially suspended in the composition in any desired crystalline or amorphous form. Such partially solubilized and partially suspended fenofibrate compositions can be prepared by adding solids of fenofibrate of desired form and particle size. For example, micronized crystalline fenofibrate having an average particle size of less than 30 microns, nanosized crystalline fenofibrate having an average particle size of less than 1 micron or meshed amorphous fenofibrate may be added to the composition. Such micronized or nanosized fenofibrate particles can be obtained by precipitation or size reduction techniques well-known in the art. In addition, partially suspended fenofibrate compositions may be obtained from a supersaturated fenofibrate solution or by co-precipitation with an additive from a fenofibrate solution.

### IV. Dosage Forms

The pharmaceutical composition of the present invention can be prepared by mixing the active agent, the solubilizer, the surfactant, and optional additives according to methods well known in the art. Alternatively, the active agent, the solubilizer, and the surfactant may be prepared in separate dosage forms or separated within one dosage form to form a dispersion in situ upon administration and dissolution in the aqueous environment of the gastrointestinal tract.

The claimed pharmaceutical compositions can be further processed according to conventional methods known to those skilled in the art, such as lyophilization, encapsulation, compression, melting, extrusion, balling, drying, chilling, molding, spraying, spray congealing, coating, comminution, mixing, homogenization, sonication, cryopelletization, spheronization and granulation to produce the desired dosage form. Excess solvent, added to facilitate incorporation of the active agent and/or mixing of the formulation components, can be removed before administration of the pharmaceutical dosage form.

The pharmaceutical compositions can be further formulated into desirable dosage forms utilizing skills well known in the art. For example, compositions in liquid, semi-solid or paste form can be filled into hard gelatin or soft gelatin capsules using appropriate filling machines. Alternatively, the composition can also be extruded, merumerized, sprayed, granulated or coated onto a substrate to become a powder, granule or bead that can be further encapsulated or tableted with or without the addition of appropriate solidifying or binding agents. This approach also allows for the creation of a “fused mixture,” a “solid solution” or a “eutectic mixture.”

In a preferred embodiment, the pharmaceutical composition is present in a single dosage form. The dosage form(s). The dosage forms of the present invention are not limited with respect to size, shape or general configuration, and may comprise, for example, a capsule, a tablet or a caplet, or a plurality of granules, beads, powders, or pellets that may or may not be encapsulated. In addition, the dosage form may be a drink or beverage solution or a spray solution that is administered orally. Thus, for example, the drink or beverage solution may be formed by adding a therapeutically effective amount of the composition in, for example, a powder or liquid form, to a suitable beverage, e.g., water or juice.

The compositions and dosage forms of the current invention may be immediate release, releasing the active agent and/or excipients in an uncontrolled fashion, or may be controlled release. Included in the term “controlled release” are dosage forms or compositions which release the drug and/or excipients with various release profiles such as extended or sustained release, delayed release, pulsitile release, or combinations of the above such as multi-stage release achieved by a combination of delayed release compositions with variable delay times.

A preferred dosage form is a capsule containing a composition as described herein. The capsule material may be either hard or soft and is generally made of a suitable compound such as gelatin, starch or a cellulosic material. As is known in the art, use of soft gelatin capsules places a number of limitations on the compositions that can be encapsulated. See, for example, Ebert (1978), “Soft Elastic Gelatin Capsules: A Unique Dosage Form,” Pharmaceutical Technology 1(5). Two-piece hard gelatin capsules are preferably sealed, such as with gelatin bands or the like. Preparation of various types of pharmaceutical formulations are described, for example, in Remington: The Science and Practice of Pharmacy, Nineteenth Edition. (1995) cited supra and Ansel et al., Pharmaceutical Dosage Forms and Drug Delivery Systems, 6th Ed. (Media, PA: Williams & Wilkins, 1995).

### V. Utility and Administration

The pharmaceutical compositions and dosage forms have utility in the treatment of patients that may benefit from the therapeutic administration of hydrophobic drugs. Such therapies include, for example, steroid therapy or hormone therapy. Patients suffering from any condition, disease or disorder that can be effectively treated with any of the active agents disclosed herein can benefit from the administration of a therapeutically effective amount of the pharmaceutical compositions and dosage forms described herein. An advantage of the claimed pharmaceutical composition is improvement in the oral absorption and bioavailability of the active agent thereby ensuring that the patient will in fact benefit from the prescribed therapy. The improved bioavailability of the active agent is a result of the improved dispersion of the active agent in the claimed pharmaceutical composition.

The composition may be administered in the form of a capsule wherein a patient swallows the entire capsule. Alternatively, the composition may be contained in capsule which is then opened and mixed with an appropriate amount of aqueous fluid such as water or juice to form a drink or beverage for administration of the composition. As will be appreciated, the composition need not be contained in a capsule and may be housed in any suitable container, e.g., packets, ampules, etc. Once prepared, the drink or beverage is imbibed in its entirety thus effecting administration of the composition. Preparation of the composition-containing drink or beverage may be effected by the patient or by another, e.g., a caregiver. As will be appreciated by those skilled in the art, additional modes of administration are available.

### EXPERIMENTAL

The practice of the present invention will employ, unless otherwise indicated, conventional techniques of pharmaceutical formulation, medicinal chemistry, and the like, which are within the skill of the art. Such techniques are explained fully in the literature. Preparation of various types of pharmaceutical formulations are described, for example, in Remington: The Science and Practice of Pharmacy, Nineteenth Edition. (1995) cited supra and Ansel et al., Pharmaceutical Dosage Forms and Drug Delivery Systems, 6.sup.th Ed. (Media, Pa.: Williams & Wilkins, 1995).

In the following examples, efforts have been made to ensure accuracy with respect to numbers used (e.g., amounts, temperature, etc.) but some experimental error and deviation should be accounted for. Unless indicated otherwise, temperature is in degrees C. and pressure is at or near atmospheric. All components were obtained commercially unless otherwise indicated. “Tweene®” and “Arlacel®” components are available from ICI Americas, Wilmington, Del. “Cremophor®” components are available from BASF Corp., Mount Olive, N.J. “Imwitor®” and “Miglyol®” components are available from Huls A G, Herne, Germany. “Lauroglycol®,” “Labrafil®,” “Labrasol®,” “Transcutol®,” “Maisine®” and “Capryol®” components are available from Gattefosse S A, Saint Priest, France. “Eastman®” components are available from Eastman Chemical Products, Inc., Kingsport, Tenn. “Captex®,” and “Capmul®” components are available from Karlshamns USA, Inc., Columbus, Ohio. “Incrocas” and “Crovol” components are available from Croda, Inc., Parsippany, N.J.

The solubility of drug substances in the compositions was determined using conventional techniques. For example, solubility was in some cases determined gravimetrically by incrementally adding drug until the composition could no longer solubilize additional added drug. Solubility could also be determined by equilibration of the composition with excess drug during gentle mixing at a controlled temperature (25±0.5° C.), centrifugation of the resulting mixture (15 min at 15,000×g; Beckmann Microfuge Lite), and assay of the clear supernatant.

The dispersibility of the composition was determined by diluting the composition in an aqueous medium such as water, simulated gastric fluid, or simulated intestinal fluid, at a selected dilution factor, preferably 10× to 1000×, most preferably 100×. The dilution was then gently mixed, for example with a rotator at 10 rpm, at an appropriate controlled temperature (typically 37° C.). After a selected duration (typically 1 hour, but any physiologically realistic duration could be appropriate), the aqueous phase was sampled, taking care not to include undispersed oil globules, or non-uniformly dispersed particulates. In some cases, the aqueous phase was filtered through Nylon or Tuffryn® membrane filters with the appropriate nominal pore size (Whatman or Gelman). In all cases, the initial 1-3 ml of filtrate were discarded, and the absence of significant filter absorption was confirmed by filtration of standard solutions of known active agent or vitamin E substance concentration in the appropriate matrix, collection of the filtrate, and assay of the filtrate to confirm that there was no change in drug concentration. Other techniques to characterize the extent of dispersion could also be used, such as centrifugation to separate larger particles from the uniform aqueous dispersion.

The aqueous phase sample or filtrate was then diluted in an appropriate solvent (typically acetonitrile or methanol; HPLC grade), and assayed for active agent or solubilizer content.

Assay for vitamin E substance content in most cases was by UV spectrophotometry with quantification at a wavelength of 291 nm for tocopherol and 285 nm for tocopherol acetate tocopherol succinate, and tocopherol polyethyleneglycol succinate. Samples were diluted 100× in methanol, then scanned in a quartz cuvette using an Agilent 8453 UV/Vis Spectrophotometer. Calibration was by linear regression of absorbance at the indicated wavelengths with standards of the relevant Vitamin E substance of known concentration. Standards of the drugs or other excipients present in the composition at the expected concentrations were also scanned to confirm selectivity.

In cases where the active agent or other excipient caused significant interference at the 285-291 nm wavelengths, assay for Vitamin E substances was by reversed phase HPLC using a Symmetry C 183.6×150 mm column, 5μ with a mobile phase of Methanol 98/2% v/v and detection at 285 nm.

Assay of the active agents was by reversed-phase HPLC with the column indicated above, a mobile phase of acetonitrile/water 63/57% v/v, and detection at 204 nm.

Particle size of aqueous dispersions was determined using a Nicomp 380 ZLS laser-scattering particle sizer (Particle Sizing Systems), with a He—Ne laser at 632.8 nm, fixed 90° angle, interrupter at 13.5°, and maximum count rate 5 MHz.

### Example 1

This example shows the solubilization and dispersion behavior of a composition including a pregnane steroid, progesterone, a vitamin E substance (dl-alpha-tocopherol, Spectrum Chemicals) and a surfactant (polyoxyl 35 castor oil USP/NF, Cremophor EL, BASF). The compositions shown in the tables below were prepared by combining the components and mixing gently at room temperature.

TABLE 1-1

Compositions

| Component | 1-1 | 1-2 | 1-3 | 1-4 |
|---|---|---|---|---|
| dl-alpha | 70% | 68.25% | 63% | 60% |
| tocopherol | | | | |
| Polyoxyl 35 | 30% | 29.25% | 27% | 26% |
| Castor Oil | | | | |
| Progesterone | 0% | 2.5% | 10% | 15% |

Compositions were dispersed in simulated gastric fluid without enzyme (USP 23) at 100× dilution (37+0.5° C.) and mixed gently for 1 hour. At 1 hour, the dispersions were filtered through 0.2μ nominal pore size Nylon filters, then the filtrate was diluted 100× in methanol and assayed for progesterone by HPLC and for tocopherol content by UV/Vis spectrophotometry. Results are shown in Table 1-2 below.

TABLE 1-2

| | Drug | | Fraction of | |
|---|---|---|---|---|
| | Loading in | Dispersion | Solubilizer | Fraction of Drug |
| No. | Concentrate | Appearance | Dispersed | Dispersed |
| 1-1 | 0 | Non-uniform | 14% | N/A |
| | | with large oil | | |
| | | globules and | | |
| | | visible | | |
| | | particulates | | |
| 1-2 | 25 mg/g | Non-uniform | 30% | 37% |
| | | with large oil | | |
| | | globules and | | |
| | | visible | | |
| | | particulates | | |
| 1-3 | 100 mg/g | Non-uniform | 41% | 36% |
| | | with a few large | | |
| | | globules | | |
| 1-4 | 150 mg/g | Uniform milky | 63% | 62% |
| | | dispersion | | |

The results in Table 1-2 show that increasing the drug loading from 0 to 15% unexpectedly improves the dispersibility of the formulation. Without the drug, the composition does not disperse readily with most of the solubilizer present in separate oily globules. With the addition of the active agent, the dispersibility of the formulation is improved such that the fraction of drug dispersed significantly increases with increasing drug loading. The fraction of drug present as a very fine (<0.2μ) dispersion increases from −37% at 25 mg/g drug loading to 62% at 15% drug loading. The improved dispersibility is also shown by the increase in the fraction of the solubilizer dispersed as a fine dispersion, increasing from 14% without drug to 63% with 150 mg/g drug.

### Example 2

This example shows the solubilization and dispersion of a pregnane steroid, progesterone, in compositions consisting of vitamin E substances (dl-alpha-tocopherol, Spectrum Chemicals; or d-alpha-tocopherol, Archer Daniels, Midland Company), a surfactant (polyoxyl 35 castor oil USP/NF, Cremophor EL, BASF), and a low-molecular weight alcohol (dehydrated alcohol, USP/NF, Quantum). The compositions shown in the tables below were prepared by combining the components and mixing gently at room temperature.

TABLE 2-1

Compositions

| Component | 2-1 | 2-2 | 2-3 | 2-4 |
|---|---|---|---|---|
| dl-alpha | 65% | 54% | — | — |
| tocopherol | | | | |
| d-alpha | — | — | 65% | 54% |
| tocopherol | | | | |
| Polyoxyl 35 | 28% | 23% | 28% | 23% |
| Castor Oil | | | | |
| Ethanol | 7% | 6% | 7% | 6% |
| Progesterone | 0% | 17.5% | 0% | 17.5% |

Compositions were dispersed in simulated gastric fluid without enzyme (USP 23) at 100× dilution (37.+−0.5° C.) and mixed gently for 1 hour. At 1 hour, the dispersions were filtered through 0.2μ nominal pore size Nylon filters, then the filtrate was diluted 100× in methanol and assayed for tocopherol content by UV/Vis spectrophotometry and progesterone content by HPLC. The particle size distribution of the dispersions was independently determined by laser scattering with a Nicomp particle size analyzer for confirmation. Results are shown in Table 2-2 below.

TABLE 2-2

| | | | | Fraction of | Volume Fraction of | Fraction of |
|---|---|---|---|---|---|---|
| | | | | Solubilizer | Particles <0.2μ, | Drug |
| | Vitamin E | | Dilution | Dispersed in | by laser | Dispersed in |
| No. | Substance | Drug | Appearance | Filtrate <0.2μ | scattering | Filtrate <0.2μ |
| 2-1 | dl-alpha- | 0 mg/g | Non-uniform | 9% | N/A* | — |
| | tocopherol | | Large globules | | | |
| | | | and particles in | | | |
| | | | cloudy solution | | | |
| 2-2 | dl-alpha- | 175 mg/g | Fine uniform | 66% | 79% | 100% |
| | tocopherol | | dispersion | | | |
| 2-3 | d-alpha- | 0 mg/g | Non-uniform | 10% | N/A* | — |
| | tocopherol | | Large globules | | | |
| | | | and particles in | | | |
| | | | cloudy solution | | | |
| 2-4 | d-alpha- | 175 mg/g | Fine uniform | 83% | 81% | 100% |
| | tocopherol | dispersion | | | | |

*Particle size cannot be accurately determined for non-uniform samples with very large particles.

The results in Table 2-2 show that not only is the drug readily soluble in the vitamin E based composition, but the presence of the drug dramatically improves the dispersibility of the composition upon aqueous dilution. Without the drug, the composition does not form a fine dispersion and only ˜10% of the vitamin E is incorporated in particles <0.2μ. With progesterone, the compositions form a very fine uniform dispersion, with ˜80% of the total vitamin E in particles <0.2μ. The assay for progesterone in the filtered dispersions shows that the drug is preferentially concentrated in these very small particles with nominal diameter <0.2μ.

### Example 3

This example shows the solubilization and dispersion of an androstane steroid ((DHEA, Sigma Chemicals), in compositions consisting of vitamin E substances (dl-alpha-tocopherol, Spectrum Chemicals; or d-alpha-tocopherol, Archer Daniels Midland Company), a surfactant (polyoxyl 35 castor oil USP/NF, Cremophor EL, BASF), and a low-molecular weight alcohol (dehydrated alcohol, USP/NF, Quantum). The compositions shown in the tables below were prepared by combining the components and mixing gently at room temperature. The corresponding placeboes (without drug) are described in Example 2, compositions 2-1 and 2-3.

TABLE 3-1

Compositions

| Component | 3-1 | 3-2 |
|---|---|---|
| dl-alpha tocopherol | 54% | — |
| d-alpha tocopherol | — | 54% |
| Polyoxyl 35 Castor Oil | 23% | 23% |
| Ethanol | 6% | 6% |
| DHEA | 17.5% | 17.5% |

Compositions were dispersed in simulated gastric fluid without enzyme (USP 23) at 100× dilution (37.+−.0.5° C.) and mixed gently for 1 hour. At 1 hour, the dispersions were filtered through 0.2μ nominal pore size Nylon filters, then the filtrate was diluted 100× in methanol and assayed for tocopherol content by UVN is spectrophotometry. Results are shown in Table 3-2 below.

TABLE 3-2

| | | | | Fraction of |
|---|---|---|---|---|
| | Vitamin E | | Dilution | Solubilizer |
| No. | Substance | DHEA | Appearance | Dispersed |
| 2-1 | dl-alpha | 0 mg/g | Non-uniform | 9% |
| | tocopherol | | Complete phase | |
| | | | separation with | |
| | | | large oil globules | |
| 3-1 | dl-alpha | 175 mg/g | Fine, uniform | 68% |
| | tocopherol | | dispersion | |
| 2-3 | d-alpha | 0 mg/g | Non-uniform | 10% |
| | tocopherol | | Large globules in | |
| | | | cloudy solution | |
| 3-2 | d-alpha | 175 mg/g | Fine, uniform | 70% |
| | tocopherol | | dispersion | |

The results in Table 3-2 show that, as with the pregnane steroid in example 2, the addition of the androstane steroid, dehydroepiandrosterone DHEA, dramatically improves the formation of a fine dispersion of the composition resulting in compositions with very high drug loading, which are then readily dispersed in aqueous media.

### Example 4

This example shows the solubilization and dispersion using Vitamin E/surfactant compositions for additional model steroids: an androstane steroid, finasteride; and a cholane steroid, ursodiol. The compositions shown in the tables below were prepared by combining the components and mixing gently at room temperature.

TABLE 4-1

| | Compositions | | |
|---|---|---|---|
| Component | 4-1 | 4-2 | 4-3 |
| dl-alpha tocopherol | 40.5% | 40% | 38% |
| Polyoxyl 35 Castor | 49.5% | 49% | 46% |
| Oil | | | |
| Ethanol | 10% | 5% | 5% |
| Finasteride | 0% | 1.1% | — |
| Ursodiol | 0% | — | 5.9% |

Compositions were dispersed in simulated gastric fluid without enzyme (USP 23) at 100× (37+0.5° C.) and mixed gently for 1 hour. At 1 hour, the dispersions were filtered through 0.2μ nominal pore size Nylon filters and the filtrate diluted 100× in methanol and assayed for tocopherol content by UV/Vis spectrophotometry. Results are shown in Table 4-2 below.

TABLE 4-2

| | | | Fraction of Solubilizer |
|---|---|---|---|
| No. | Drug | Dilution Appearance | Dispersed |
| 4-1 | No drug | Non-uniform, large | 40% |
| | | particles and globules | |
| 4-2 | Finasteride | Fine, uniform | 86% |
| | | dispersion | |
| 4-3 | Ursodiol | Fine, uniform | 93% |
| | | dispersion | |

The results in Table 4-2 show that, as with the other steroids tested, the incorporation of the steroid active agent has a critical role in achieving good dispersion of the composition upon aqueous dilution.

### Example 5

This example shows the solubilization and dispersion of progesterone in compositions containing two different tocopherol esters (d-alpha-tocopherol acetate and d-alpha-tocopherol succinate, Archer Daniels Midland Company). The compositions shown in the tables below were prepared by combining the components and mixing gently at room temperature.

TABLE 5-1

Compositions

| Component | 5-1 | 5-2 | 5-3 | 5-4 |
|---|---|---|---|---|
| d-alpha | 79% | 71% | — | — |
| tocopherol | | | | |
| acetate | | | | |
| d-alpha | — | — | 68% | 62% |
| tocopherol | | | | |
| succinate | | | | |
| Polyoxyl 35 | 14% | 13% | 29% | 27% |
| Castor Oil | | | | |
| Ethanol | 7% | 6% | 3% | 3% |
| Progesterone | 0% | 10% | 0% | 8% |

Compositions were dispersed in simulated gastric fluid without enzyme (USP 23) at 100× dilution (37.+−.0.5° C.) and mixed gently for 1 hour. At 1 hour, the dispersions were filtered through 0.2μ nominal pore size Nylon filters, then the filtrate was diluted 100× in methanol and assayed for tocopherol succinate content by UV/Vis spectrophotometry. Results are shown in Table 5-2 below.

TABLE 5-2

| | | | | Fraction of |
|---|---|---|---|---|
| | Vitamin E | | Dilution | Solubilizer |
| No. | Substance | Drug | Appearance | Dispersed |
| 5-1 | d-alpha | 0 mg/g | Non- | 28% |
| | tocopherol | | homogeneous | |
| | acetate | | dispersion, Large | |
| | | | globules in | |
| | | | cloudy solution | |
| 5-2 | d-alpha | 100 mg/g | Fine uniform | 72% |
| | tocopherol | | dispersion | |
| | acetate | | | |
| 5-3 | d-alpha | 0 mg/g | Non- | 66% |
| | tocopherol | | homogeneous | |
| | succinate | | dispersion, Large | |
| | | | globules in | |
| | | | cloudy solution | |
| 5-4 | d-alpha | 84 mg/g | Fine uniform | 99% |
| | tocopherol | | dispersion | |
| | succinate | | | |

The results in Table 5-2 show that both tocopherol esters have excellent solubilizing capacity for the steroid and allow for very high drug loading. The results also show that the steroid is critical to achieving adequate dispersion of the composition. Without the steroid, the composition is visibly non-uniform with the bulk of the composition in large particles or globules (only 30%<0.45μ). With the steroid drug, the composition is readily dispersed with more 70% of the particles in a fine dispersion which passes through the 0.45μ filter.

### Example 6

This example shows the effect of solubilization and dispersion of progesterone in compositions with varying surfactants and surfactant levels. The vitamin E substances are d-alpha tocopherol or d-alpha tocopherol acetate (both from Archer Daniels Midland) with the following surfactants: polyoxyl 35 castor oil (Cremophor EL, BASF); caprylocaproyl macrogolglycerides (Labrasol, Gattefosse); polysorbate 80 (Tween 80, ICI), medium chain monoglycerides (Capmul MCM, Abitec), and tocopherol polyethyleneglycol 1000 succinate (Vitamin E-TPGS, Eastman). The compositions shown in the tables below were prepared by combining the components and mixing gently at room temperature.

TABLE 6-1

| | Composition | | | |
|---|---|---|---|---|
| | Tradename, | | | |
| Component | Source | 6-1 | 6-2 | 6-3 |
| dl-alpha tocopherol | Vitamin E USP, | 77% | 29% | — |
| | Spectrum | | | |
| dl-alpha tocopherol | Vitamin E 6-100, | — | — | 85% |
| acetate | ADM | | | |
| Dehydrated | Ethanol, 200 | 15% | 3% | — |
| Alcohol | proof, Quantum | | | |
| Polyoxyl 35 Castor | Cremophor EL, | 9% | — | — |
| Oil | BASF | | | |
| Caprylocaproyl | Labrasol, | — | 68% | — |
| macrogolglycerides | Gattefosse | | | |
| Polysorbate 80 | Tween 80, ICI | — | — | 5% |
| Medium chain | Capmul MCM, | | | 7.5% |
| monoglycerides | Abitec | | | |
| Tocopherol | Vitamin E- | — | — | 2.5% |
| polyethylene | TPGS, Eastman | | | |
| glycol 1000 | | | | |
| succinate | | | | |
| Progesterone | N/A | 0% | 0% | 0% |

TABLE 6-2

| | Composition | | | |
|---|---|---|---|---|
| | Tradename, | | | |
| Component | Source | 6-4 | 6-5 | 6-6 |
| dl-alpha tocopherol | Vitamin E USP, | 59% | 27% | — |
| | Spectrum | | | |
| dl-alpha tocopherol | Vitamin E 6-100, | — | — | 77.8% |
| acetate | ADM | | | |
| Dehydrated | Ethanol, | 7% | 3% | — |
| Alcohol | Quantum | | | |
| Polyoxyl 35 Castor | Cremophor EL, | 12% | — | — |
| Oil | BASF | | | |
| Caprylocaproyl | Labrasol, | — | 63% | — |
| macrogolglycerides | Gattefosse | | | |
| Polysorbate 80 | Tween 80, ICI | — | — | 4.6% |
| Medium chain | Capmul MCM, | | | 6.9% |
| monoglycerides | Abitec | | | |
| Tocopherol | Vitamin E- | — | — | 2.3% |
| polyethylene | TPGS, Eastman | | | |
| glycol 1000 | | | | |
| succinate | | | | |
| Progesterone | N/A | 22.5% | 7% | 8.5% |

Compositions were dispersed in simulated gastric fluid without enzyme (USP 23) at 100× dilution (37+0.5° C.) and mixed gently for 1 hour. At 1 hour, the bulk aqueous phase was sampled, taking care not to disturb the oily phase. The sample was then diluted 100× in methanol and assayed for tocopherol and drug content by UV/Vis spectrophotometry or HPLC. Results are shown in Table 6-3 below.

TABLE 6-3

| | | Vitamin | | | Fraction of | Fraction |
|---|---|---|---|---|---|---|
| | | E:Surfactant | | Dilution | Solubilizer | of Drug |
| No. | Surfactant(s) | Ratio | Drug | Appearance | Dispersed | Dispersed |
| 6-1 | Polyoxyl 35 Castor | 9:1 | 0 mg/g | Complete | 0% | — |
| | Oil | | | phase | | |
| | | | | separation, | | |
| | | | | visible oily | | |
| | | | | layer, | | |
| | | | | essentially | | |
| | | | | clear | | |
| | | | | aqueous | | |
| | | | | phase | | |
| 6-4 | Polyoxyl 35 Castor | 9:1 | 225 mg/g | Hazy | 69% | 65% |
| | Oil | | | dispersion | | |
| | | | | with a few | | |
| | | | | large | | |
| | | | | visible | | |
| | | | | globules | | |
| 6-2 | Caprylocaproyl | 3:7 0 | mg/g | Complete | 17% | — |
| | macrogolglycerides | | | phase | | |
| | | | | separation | | |
| | | | | with visible | | |
| | | | | oily | | |
| | | | | globules, | | |
| | | | | cloudy | | |
| | | | | aqueous | | |
| | | | | phase | | |
| 6-5 | Caprylocaproyl | 3:7 | 70 mg/g | Hazy | 68% | 69% |
| | macrogolglycerides | | | dispersion | | |
| | | | | with a few | | |
| | | | | large | | |
| | | | | visible | | |
| | | | | globules | | |
| 6-3 | Polysorbate | 5.7:1 | 0 | Non | 44%^{a} | — |
| | 80/Medium chain | | | uniform, | | |
| | monoglycerides/E- | | | cloudy with | | |
| | TPGS | | | visible | | |
| | | | | particulates | | |
| 6-6 | Polysorbate | 5.7:1 | 85 mg/g | Completely | 100%^{a} | 100%^{a} |
| | 80/Medium chain | | | dispersed | | |
| | monoglycerides/E- | | | in fine, | | |
| | TPGS | | | slightly | | |
| | | | | hazy | | |
| | | | | dispersion | | |

^{a}Filtered with 0.45μ filter before assay.

The results in table 6-3 show that for all surfactants and surfactant levels, not only is the drug well solubilized in the vitamin E substance composition, but it also plays a critical role in dispersing the composition upon aqueous dilution.

### Example 7

This example shows the effect of solubilization and dispersion of progesterone in a compositions consisting of a vitamin E substance (d-alpha-tocopherol), a surfactant (polyoxyl 35 castor oil USP/NF) and various hydrophilic and hydrophobic solvents (ethanol, triethyl citrate; glycerol triacetate (triacetin)). The compositions shown in the tables below were prepared by combining the components and mixing gently at room temperature.

TABLE 7-1

Compositions

| | Tradename, | | | |
|---|---|---|---|---|
| Component | Source | 7-1 | 7-2 | 7-3 |
| dl-alpha | Vitamin E USP, | 65% | 65% | 65% |
| tocopherol | Spectrum | | | |
| Polyoxyl 35 | Cremophor EL, | 28% | 28% | 28% |
| Castor Oil | BASF | | | |
| Ethanol | Ethanol, 200 | 7% | — | — |
| | proof, Quantum | | | |
| Triethyl citrate | Triethyl citrate, | — | 7% | — |
| | Aldrich | | | |
| Triacetin | Triacetin, | — | — | 7% |
| | Eastman | | | |
| Progesterone | N/A | 0% | 0% | 0% |

TABLE 7-2

Compositions

| | Tradename, | | | |
|---|---|---|---|---|
| Component | Source | 7-4 | 7-5 | 7-6 |
| dl-alpha | Vitamin E, USP, | 59% | 59% | 59% |
| tocopherol | Spectrum | | | |
| Polyoxyl 35 | Cremophor EL, | 25% | 25% | 25% |
| Castor Oil | BASF | | | |
| Ethanol | Ethanol, 200 | 6% | — | — |
| | proof, Quantum | | | |
| Triethyl citrate | Triethyl citrate, | — | 6% | — |
| | Aldrich | | | |
| Triacetin | Triacetin, | — | — | 6% |
| | Eastman | | | |
| Progesterone | N/A | 10% | 10% | 10% |

Compositions were dispersed in simulated gastric fluid without enzyme (USP 23) at 100× dilution (37+0.5° C.) and mixed gently for 1 hour. At 1 hour, the dispersions were filtered through 0.2μ nominal pore size Nylon filters, the filtrate was then diluted 100× in methanol and assayed for tocopherol content by UV/Vis spectrophotometry. Results are shown in Table 7-3 below.

TABLE 7-3

| | | | | Fraction of |
|---|---|---|---|---|
| | | | Dilution | Solubilizer |
| No. | Solvent | Drug | Appearance | Dispersed |
| 7-1 | Ethanol | 0 mg/g | Non uniform | 18% |
| | | | dispersion with | |
| | | | large visible | |
| | | | globules | |
| 7-4 | Ethanol | 100 mg/g | Fine uniform | 69% |
| | | | dispersion | |
| 7-2 | Triacetin | 0 mg/g | Non uniform | 24% |
| | | | dispersion with | |
| | | | large visible | |
| | | | globules | |
| 7-5 | Triacetin | 100 mg/g | Fine uniform | 47% |
| | | | dispersion | |
| 7-3 | Triethyl Citrate | 0 mg/g | Non uniform | 15% |
| | | | dispersion with | |
| | | | large visible | |
| | | | globules | |
| 7-6 | Triethyl Citrate | 100 mg/g | Fine uniform | 45% |
| | | | dispersion | |

This example shows that for each of the solvents tested, the presence of the steroid drug significantly improves the dispersibility of the composition in aqueous medium.

### Example 8-23

Exemplary Compositions

| | |
|---|---|
| Component | Amount (mg) |

EXAMPLE 8

| | |
|---|---|
| dl-alpha tocopherol | 520 |
| Cremophor EL | 430 |
| DHEA | 50 |

EXAMPLE 9

| | |
|---|---|
| dl-alpha tocopherol | 55 |
| Cremophor RH40 | 45 |
| Dutasteride | 0.5 |

EXAMPLE 10

| | |
|---|---|
| dl-alpha tocopherol | 200 |
| Polysorbate 80 | 15 |
| Maisine (Glycerol monolinoleate) | 30 |
| Eplerenone | 40 |

EXAMPLE 11

| | |
|---|---|
| dl-alpha tocopherol | 300 |
| Capryol 90 (Propylene glycol monocaprylate) | 100 |
| Cremophor EL | 60 |
| Spironolactone | 200 |

EXAMPLE 12

| | |
|---|---|
| dl-alpha tocopherol | 313 |
| Cremophor EL | 256 |
| Dehydrated Alcohol | 70 |
| Progesterone | 60 |

EXAMPLE 13

| | |
|---|---|
| d-alpha tocopherol succinate | 60 |
| E-TPGS | 540 |
| PEG 8000 | 60 |
| Progesterone | 100 |

EXAMPLE 14

| | |
|---|---|
| d-alpha tocopherol succinate | 60 |
| E-TPGS | 540 |
| PEG 8000 | 60 |
| Testosterone | 100 |

EXAMPLE 15

| | |
|---|---|
| dl-alpha tocopherol | 300 |
| Cremophor RH40 | 300 |
| Coenzyme Q10 | 100 |

EXAMPLE 16

| | |
|---|---|
| dl-alpha tocopherol | 300 |
| Cremophor RH40 | 300 |
| Idebenone | 90 |

EXAMPLE 17

| | |
|---|---|
| d-alpha tocopherol | 270 |
| Alpha-tocotrienol | 2 |
| Gamma-tocotrienol | 23 |
| Cremophor RH40 | 300 |
| Idebenone | 90 |

EXAMPLE 18

| | |
|---|---|
| dl-alpha tocopherol | 80 |
| Cremophor RH40 | 400 |
| Crovol M-40 | 350 |
| Coenzyme Q10 | 100 |

EXAMPLE 19

| | |
|---|---|
| Tocoperyl polyethylene glycol 400 succinate | 200 |
| Tocopherol polyethylene glycol 1000 succinate | 100 |
| PEG 3350 | 5 |
| Bicalutamide | 50 |

EXAMPLE 20

| | |
|---|---|
| d-alpha tocopherol succinate | 250 |
| Cremophor RH40 | 50 |
| Capmul MCM | 50 |
| Simvastatin | 10 |

EXAMPLE 21

| | |
|---|---|
| d-alpha tocopherol succinate | 200 |
| Cremophor RH40 | 200 |
| Glycerol Dibehenate (Compritol 888) | 100 |
| Glycerol Distearate (Precirol) | 80 |
| Metaxalone | 300 |

EXAMPLE 22

| | |
|---|---|
| d-alpha tocopherol succinate | 100 |
| Hydroxypropyl methyl cellulose, USP | 100 |
| (Methocel K4M) | |
| Microcrystalline cellulose, USP (Avicel PH | 200 |
| 101) | |
| Polyoxyl 40 Hydrogenated Castor Oil, USP | 120 |
| (Cremophor RH 40) | |
| Polyvinyl pyrrolidone, USP (Kollidon 90F) | 45 |
| Talc, USP | 8.75 |
| Colloidal Silicon dioxide, USP (Cab-o-Sil | 1.25 |
| treated) | |
| Dehydroepiandrosterone | 100 |

EXAMPLE 23

| | |
|---|---|
| Drug-Containing Granules: | |
| Spironolactone | 100.0 |
| Butylated Hydroxy Anisole USP-NF (BHA) | 0.05 |
| Microcrystalline Cellulose USP-NF | 100.0 |
| Crospovidone USP-NF | 27.5 |
| Polyvinyl pyrrolidone USP-NF | 40.0 |
| Talc USP-NF | 4.0 |
| Colloidal Silicon dioxide USP-NF | 2.0 |
| Magnesium Stearate USP-NF | 2.0 |
| Solubilizer/Surfactant Granules: | |
| Cremophor RH40 | 300 |
| Tocopherol Polyethyleneglycol 400 succinate | 50 |
| d-alpha tocopherol succinate | 50 |
| Sodium Starch Glycolate USP-NF | 22 |
| Colloidal Silicon dioxide USP-NF | 122 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A pharmaceutical composition comprising:
a) a therapeutically effective amount of a sex hormone; and
b) an effective solubilizing amount of a solubilizer, the solubilizer comprising a vitamin E substance, a glycerol fatty acid ester, a propylene glycol fatty acid ester, an ethylene glycol fatty acid ester, a trialkyl citrate, a lower alcohol fatty acid ester, a phospholipid, ethanol, PEG, or a combination thereof,
wherein the bioavailability of the sex hormone is improved when the pharmaceutical composition is administered compared to the biovailability of the sex hormone administered without an effective solubilizing amount of the solubilizer.

2. The pharmaceutical composition of claim 1, wherein the solubilizer is a glycerol fatty acid ester.

3. The pharmaceutical composition of claim 2, wherein the glycerol fatty acid ester is a monoglyceride.

4. The pharmaceutical composition of claim 2, wherein the glycerol fatty acid ester is a diglyceride.

5. The pharmaceutical composition of claim 2, wherein the glycerol fatty acid ester is a triglyceride.

6. The pharmaceutical composition of claim 1, wherein the solubilizer is present in the composition in an amount of from about 5 wt % to about 95 wt %.

7. The pharmaceutical composition of claim 6, wherein the solubilizer is present in the composition in an amount of from about 20 wt % to about 70 wt %.

8. The pharmaceutical composition of claim 1, further comprising a surfactant.

9. The pharmaceutical composition of claim 8, wherein the surfactant comprises a polyethoxylated fatty acid, an alcohol-oil transesterification product, a transesterification product of a oil and alcohol, or a combination thereof.

10. The pharmaceutical composition of claim 9, wherein the surfactant is a polyethoxylated castor oil or polyethoxylated hydrogenated castor oil.

11. The pharmaceutical composition of claim 10, wherein the surfactant comprises polyoxyl 35 castor oil, PEG-40 hydrogenated castor oil, or a combination thereof.

12. The pharmaceutical composition of claim 8, wherein the surfactant is present in the composition in an amount of up to about 80 wt %.

13. The pharmaceutical composition of claim 12, wherein the surfactant is present in the composition in an amount of from about 10 wt % to about 50 wt %.

14. The pharmaceutical composition of claim 1, wherein the pharmaceutical composition is a liquid, semi-solid, or solid.

15. The pharmaceutical composition of claim 14, wherein the composition is formulated into a capsule.

16. The pharmaceutical composition of claim 1, wherein the solubilizer enhances the bioabsorption of the sex hormone.

17. The pharmaceutical composition of claim 1, wherein the solubilizer increases the rate and extent of absorption of the sex hormone.

18. The pharmaceutical composition of claim 1, wherein the solubilizer provides for a more rapid onset of therapeutic action, better bioperformance characteristics, or a combination thereof of the sex hormone.

19. The pharmaceutical composition of claim 1, wherein the sex hormone is progesterone, testosterone, or an ester thereof.

20. The pharmaceutical composition of claim 19, wherein the sex hormone is testosterone enanthate, testosterone propionate, testosterone undecanoate, testosterone palmitate, or a combination thereof.

21. The pharmaceutical composition of claim 19, wherein the testosterone ester is testosterone enanthate.

22. The pharmaceutical composition of claim 19, wherein the testosterone ester is testosterone proprionate.

23. The pharmaceutical composition of claim 19, wherein the testosterone ester is testosterone undecanoate.

24. The pharmaceutical composition of claim 19, wherein the testosterone ester is testosterone palmitate.

25. The pharmaceutical composition of claim 1, wherein upon dilution of the composition, the active agent increases the extent of dispersion of the vitamin E substance by at least 20% relative to the dispersion of the composition without the active agent.

26. A method of providing hormone therapy in a patient comprising orally administering to a patient in need thereof a pharmaceutical composition comprising:
a) a therapeutically effective amount of sex hormone; and
b) an effective solubilizing amount of a solubilizer selected from the group consisting of a vitamin E substance, a glycerol fatty acid ester, a propylene glycol fatty acid ester, an ethylene glycol fatty acid ester, a trialkyl citrate, a lower alcohol fatty acid ester, a phospholipid, ethanol, PEG, or a combination thereof.

27. The method of claim 26, wherein the solubilizer is a glycerol fatty acid ester.

28. The method of claim 27, wherein the glycerol fatty acid ester is a monoglyceride.

29. The method of claim 27, wherein the glycerol fatty acid ester is a diglyceride.

30. The method of claim 27, wherein the glycerol fatty acid ester is a triglyceride.

31. The method of claim 26, wherein the solubilizer is present in the composition in an amount of from about 5 wt % to about 95 wt %.

32. The method of claim 31, wherein the solubilizer is present in the composition in an amount of from about 20 wt % to about 70 wt %.

33. The method of claim 26, further comprising a surfactant.

34. The method of claim 33, wherein the surfactant comprises a polyethoxylated fatty acid, an alcohol-oil transesterification product, a transesterification product of a oil and alcohol, or a combination thereof.

35. The method of claim 34, wherein the surfactant is a polyethoxylated castor oil or polyethoxylated hydrogenated castor oil.

36. The method of claim 35, wherein the surfactant comprises polyoxyl 35 castor oil, PEG-40 hydrogenated castor oil, or a combination thereof.

37. The method of claim 33, wherein the surfactant is present in the composition in an amount of up to about 80 wt %.

38. The method of claim 37, wherein the surfactant is present in the composition in an amount of from about 10 wt % to about 50 wt %.

39. The method of claim 26, wherein the pharmaceutical composition is a liquid, semi-solid, or solid.

40. The method of claim 39, wherein the composition is formulated into a capsule.

41. The method claim 26, wherein bioabsorption of the sex hormone is enhanced compared to a method of administering the sex hormone without the solubilizer.

42. The method claim 26, wherein onset and duration of absorption of the sex hormone is increased compared to a method of administering the sex hormone without the solubilizer.

43. The method claim 26, wherein onset of therapeutic action is more rapid, bioperformance characteristics of the sex hormone are better, or a combination thereof compared to a method of administering the sex hormone without the solubilizer.

44. The method claim 26, wherein the sex hormone is progesterone, testosterone or an ester thereof.

45. The method of claim 44, wherein the sex hormone is testosterone enanthate, testosterone propionate, testosterone undecanoate, testosterone palmitate, or a combination thereof.

46. The method claim 44, wherein the testosterone ester is testosterone enanthate.

47. The method claim 44, wherein the testosterone ester is testosterone proprionate.

48. The method claim 44, wherein the testosterone ester is testosterone undecanoate.

49. The method of claim 44, wherein the testosterone ester is testosterone palmitate.
